(19) United States
(12) Patent Application Publication
(10) Pub. No.: US 2022/0304922 A1
(43) Pub. Date: Sep. 29, 2022

(54) ULTRASOUND PHANTOM

(71) Applicant: NORWEGIAN UNIVERSITY OF SCIENCE AND TECHNOLOGY (NTNU), Trondheim (NO)

(72) Inventors: Torjus Lines STEFFENSEN, Trondheim (NO); Carlo KRIESI, Trondheim (NO); Martin STEINERT, Trondheim (NO); Thomas LAFRENZ, Trondheim (NO); Jostein Rødseth BREDE, Trondheim (NO); Marius AUFLEM, Trondheim (NO)

(21) Appl. No.: 17/641,651

(22) PCT Filed: Sep. 9, 2020
(86) PCT No.: PCT/EP2020/075249

§ 371 Date: Mar. 9, 2022

(30) Foreign Application Priority Data
Sep. 10, 2019 (GB) 1913066.5

(51) Int. Cl. A61K 9/00; A61K 47/06

(52) U.S. Cl. CPC A61K 9/0014; A61K 47/06; A61Q 19/00

(57) ABSTRACT

An ultrasound phantom comprising a hydrocarbon gel layer encased in a silicone sleeve, said hydrocarbon gel being doped with inorganic powder.

This invention relates to an ultrasound phantom, in particular, an ultrasound phantom comprising a doped hydrocarbon gel and a silicone sleeve. Ideally, the ultrasound phantom comprises a doped hydrocarbon gel encased within a protective silicone sleeve.

### BACKGROUND OF INVENTION

Ultrasound is a valuable diagnostic technique. The high accuracy of quantitative measurement of ultrasound speed or attenuation in tissue using an ultrasound CT device is very important in determining patient health.

For various reasons it is important to have an ultrasound phantom, i.e. a material that mimics the acoustic properties of the body. A phantom can be used to calibrate or recalibrate an ultrasound device. More importantly, it can be used in training of ultrasonographers and doctors. Ultrasound phantoms are used as simulation education tools for repeated practice of ultrasound-related skills, including the location of embedded foreign bodies, identifying abscesses, and practicing ultrasound-guided vascular access.

Phantoms are designed to offer the same feel as human tissue so that when subjected to medical procedures such as injections the physician experiences the same feel as if treating a real patient.

There are many ultrasound phantoms on the market. For accuracy control, a phantom is required to mimic the acoustic properties of the body and to maintain the same quantitative value over extended time periods. Many current phantoms are based on aqueous gels such as agar or gelatin. Such gels are good mimics of the human body but are susceptible to drying out. Also, being biological products they are susceptible to microorganism degradation.

Alternatives to aqueous gels include urethane rubbers and polyvinyl alcohol cryogels.

In WO2009/010898, an ultrasound phantom is described which is based on latex layer and an aqueous gel layer. This phantom is said to offer excellent mimicry during a needle insertion procedure.

In Vieira Ultrasound in Med & Biol. Vol. 39, No. 12, p2477-2484, paraffin gel-wax tissue mimicking materials are mentioned which may contain glass microspheres. This phantom is designed to mimic a breast.

The present inventors sought a non-aqueous phantom with a long shelf life. Ideally, such a phantom should not be at risk of microorganism degradation. The inventors have appreciated that hydrocarbon gel based waxes are ideal non-aqueous ultrasound phantoms but are prone to damage and tearing and do not therefore have a desirable shelf life. As these products are expensive, there remains a need to solve the problem of shelf life whilst also avoiding water and biologically degradable substances.

### SUMMARY OF INVENTION

Viewed from one aspect the invention provides an ultrasound phantom comprising a hydrocarbon gel layer encased in a protective silicone sleeve, said hydrocarbon gel being doped with inorganic powder.

Alternatively viewed, the invention provides ultrasound phantom comprising a hydrocarbon gel layer doped with inorganic powder disposed within an open container, such as a plastic container, said hydrocarbon gel layer having a lower surface in contact with the base of the container and an upper surface, wherein a protective silicone layer covers, such as entirely covers, the upper surface of said hydrocarbon gel layer.

Alternatively viewed, the invention provides ultrasound phantom comprising a hydrocarbon gel layer doped with inorganic powder disposed within an open housing, such as a plastic container, said hydrocarbon gel layer having a lower surface in contact with the base of the housing and an upper surface, wherein a protective silicone layer covers, such as entirely covers, the upper surface of said hydrocarbon gel layer.

Viewed from another aspect the invention provides an ultrasound phantom comprising a hydrocarbon gel layer completely encased in a protective silicone sleeve, said hydrocarbon gel being doped with inorganic powder.

Viewed from another aspect the invention provides the use of a phantom as hereinbefore defined in ultrasonography and/or as a mimic for needle insertion.

Viewed from another aspect the invention provides a process for the preparation of an ultrasound phantom as herein before defined comprising preparing a hydrocarbon gel doped with inorganic powder and completely encasing the same in a protective silicone sleeve.

### DETAILED DESCRIPTION OF INVENTION

This invention relates to an ultrasound phantom that can be used to train ultrasonographers and which can mimic body tissues, e.g. during needle insertion. The phantom can be shaped into any desired shape, such as a body part shape, but conveniently it is provided as a cuboid. If the phantom is designed to mimic a particular body structure, then the phantom may be prepared in the shape of that body structure, such as breasts, testes and so on.

### Hydrocarbon Gel Layer

The phantom comprises a hydrocarbon gel. The hydrocarbon gel is a combination of a hydrocarbon, such as paraffin or mineral oil, and a gelling agent. The hydrocarbon is typically one that is liquid at room temperature (23° C.) or is a low melting point solid at room temperature, e.g. having a melting point up to 100° C., e.g. a wax. Suitable hydrocarbons include mineral oil, paraffin, petroleum jelly and the like. The use of paraffin is preferred.

The gelling agent is not biological. In a preferred embodiment the gelling agent is a polyolefin, such as a polyolefin copolymer such as one used in the manufacture of candles. A preferred polymer is an alpha-olefin copolymer with styrene. These polymers are often block copolymers. Specific copolymers of interest are ethylene/propylene/styrene copolymers or butylene/ethylene/styrene copolymers. Mixtures of such polymers may also be used.

Suitable gelling agents are available commercially such as Penreco CP9000. The gelling agents and the gels that they form with the hydrocarbon are often used in candle manufacture. They are described in documents such as U.S. Pat. No. 5,578,089A, 5,879,694A, 6,066,329A, and 6,471,731B1.

The gelling agent may therefore be a block copolymer with a rigid block and an elastomeric block, such as a polystyrene/polyolefin block copolymer. The block copolymer is preferably selected from the group consisting of polystyrene/ethylene-propylene copolymer, polystyrene/ethylene-butadiene copolymer, and polystyrene/butadiene copolymer.

Alternatively, the block copolymer is selected from the group consisting of polystyrene/polyester, polyether/polyamide, polyether/polyester, polyester/polyimide, polyether/polyurethane, polyester/polyurethane, poly(ethylene oxide)/poly(propylene oxide), nylon/rubber, and polysiloxane/polycarbonate.

The amount hydrocarbon in the hydrocarbon gel as a whole (and hence in the hydrocarbon gel layer as a whole) is preferably at least 80 wt % such as 85 to 98 wt %, more preferably 90 to 98 wt %, especially 92 to 97 wt %. The use of about 95 wt % hydrocarbon is preferred.

The amount of gelling agent in the hydrocarbon gel as a whole may be 2.0 to 15 wt %, such as 2.0 to 10 wt %, preferably 3.0 to 8.0 wt %.

The amount of gelling agent added can be altered to change the stiffness of the material (also described as the bulk modulus), in turn affecting both the speed of sound and the acoustic impedance. Acoustic impedance affects the ultrasound image in important ways as it determines the amount of reflection between different materials or structures.

Increased stiffness can also make the phantom feel more natural e.g. in terms of needle feel, touch, etc.

In order to allow this material to mimic an animal body in terms of its response to ultrasound, it is required that an amount of inorganic powder is added to the hydrocarbon gel material. The doping of the hydrocarbon gel layer (i.e. the introduction of an “impurity” substance) improves echogenicity. The amount of such powder may be 0.1 to 5.0 wt % of the hydrocarbon gel, such as 0.5 to 2.0 wt %.

Inorganic powders of interest include metal oxides such as alumina, glass, and titanium dioxide or graphite. Most preferably however the inorganic powder is graphite or titanium white (titanium dioxide). The use of titanium dioxide is especially preferred as it has an effect of whitening the material and hence giving more flexibility to develop a skin like colour through the use of a dye.

Graphite and titanium dioxide are biologically inert and safe to handle.

The particle size of the inorganic particles may be in the range of 0.5 to 100 μm, such as 0.1 to 50 μm, more preferably 25 to 37 μm. The presence of inorganic powder not only increases the echogenicity of the material but renders it more natural in terms of its ultrasound properties.

The thickness of the hydrocarbon gel layer can be tailored to match requirements. The thickness is defined as the smallest dimension in any 3-D structure. Typically a hydrocarbon gel layer may be 0.25 to 2.0 cm in thickness, such as 0.75 to 1.5 cm.

It is preferred if the hydrocarbon gel layer contains less than 5 wt % water, especially less than 1.0 wt % water, especially is non-aqueous.

### Silicone Sleeve and Silicone Layer

We have found that the hydrocarbon gel is not suitable for a commercial phantom by itself. Exposed to the environment, it is liable to tear and damage. It is therefore required that the hydrocarbon gel is protected such as encased in a protective silicone sleeve or protected via a housing/container with a silicone layer covering. That silicone sleeve or silicone layer is one that prevents damage to the hydrocarbon gel layer but also has properties that mimic the body tissues. Silicones are polysiloxanes.

Preferably therefore the protective silicone sleeve or silicone layer comprises a silicone, such as a silicone rubber (such as one from the Smooth-On Ecoflex™ series). The silicone rubber may be one that cures. It may have a Shore hardness of 00-5 to 00-50, preferably 00-10 to 00-30. Suitable silicone rubbers are available commercially.

The silicone sleeve or silicone layer itself preferably comprises at least 70 wt % silicone, such as at least 80 wt % silicone, e.g. at least 90 wt % silicone. In one embodiment, the silicone sleeve or layer can be dusted with talc after manufacture to prevent tackiness or stickiness of the silicone rubber, resulting in a more natural sensation of touch.

In order to make sure that the silicone sleeve or silicone layer mimics animal tissue, it is preferred if the silicone sleeve or silicone layer is combined with an inorganic powder. Suitable powders include those mentioned above in connection with the hydrocarbon gel layer, e.g. alumina, glass, and titanium dioxide or graphite. The use of ceramic powder, such as alumina or titanium white powder is most preferred. Graphite is preferably avoided as this makes the silicone black which is not favoured for the sleeve.

There may be 0.5 to 2.0 wt % of powder present in the silicone sleeve or silicone layer, such as approximately 1% wt.

It is also preferred if the silicone sleeve or silicone layer includes up to 30% wt glycerine (C₃H₈O₃), such as 10 to 25 wt % glycerine. The addition of glycols is also possible such as 5.0 to 30 wt % glycols. If both glycerine and glycols are present, the total amount of these components is preferably in the range of 5.0 to 30 wt %. Preferred glycols include propylene glycol.

The presence of glycerine or a glycol increases the speed of sound through the material, reducing speed displacement error. It also reduces bulk material cost.

It is possible to add pigments to the silicone sleeve or silicone layer so that it has a natural skin like colour.

In one embodiment, the silicone sleeve preferably completely encapsulates the hydrocarbon gel layer so that this layer is not exposed.

In a second embodiment, the hydrocarbon gel layer is disposed within an open housing or container which partially protects the hydrocarbon gel layer. Ideally, the hydrocarbon gel layer fills the housing or container such that the lower surface of the hydrocarbon gel layer contacts the base of the container or housing. It is also preferred if the edges of the hydrocarbon gel layer contact the sides of the container or housing. As the hydrocarbon gel layer may be added to the container in liquid form, it will naturally fill the container.

It may be that the container or housing has, for example, an adhesive to secure the hydrocarbon gel layer within the container or housing. In that scenario the lower surface of the gel layer is still considered to be in contact with the housing or container even if an adhesive layer is present.

The upper surface of the hydrocarbon gel layer is exposed and is covered using the silicone layer. Ideally, the container or housing is filled such that the top of the housing or container and the upper surface of the gel layer within form an essentially flat surface onto which the silicone layer can be applied. It is however possible for the silicone layer to cover the hydrocarbon gel layer (such as entirely cover the hydrocarbon gel layer) within the housing or container.

The hydrocarbon gel layer may protrude from the container such that the silicone layer needs to protect parts of the exposed sides of the gel layer as well as its upper surface. The skilled person can devise solutions in which all parts of the hydrocarbon gel layer are protected by the container and the silicone layer.

Typically however, the silicone layer protects the exposed upper surface of the hydrocarbon gel layer. The housing or container therefore protects the bottom and sides of the hydrocarbon gel layer whilst the silicone layer protects the top of the hydrocarbon gel layer.

In theory, the hydrocarbon gel layer may not completely cover the base of the container or housing such that there are gaps between the edges of the container or housing and the hydrocarbon gel layer therein. In that scenario, the silicone layer could cover both the upper surface of the hydrocarbon gel layer and the sides of the hydrocarbon gel layer, e.g. as shown below:

In this embodiment, it is in theory possible to remove the container sides such that only the bottom of the container remains as the silicone layer protects the other surfaces of the hydrocarbon gel layer.

In one embodiment the housing or container is provided with a flange in order to provide a surface onto which to adhere the silicone layer. The housing or container can therefore contain a flange parallel to the top of the housing or container which provides a surface to which a silicone layer can adhere. As shown in FIG. 8 the silicone layer can wrap around the flanges to improve adhesion. The flange may be positioned at opposite ends of the housing or container or could extend around the entire top of the housing or container. Any suitable flange design is possible.

The housing or container is ideally made from plastic such as a vacuum formed plastic housing or container.

Ideally the silicone layer contacts both the top of the housing or container and the hydrocarbon gel layer. It may therefore seal or close the housing or container.

The phantom of the invention therefore has excellent ultrasound reproduction from hydrocarbon gel, and durability from silicone sleeve, silicone layer and/or housing/container. The phantom also has a lifelike feel. When compressed for example, the phantom feels tissue like. The phantom therefore also provides realistic feedback while guiding a needle through the material, avoiding high friction on needle from thick layers of rubber.

The outer appearance of the phantom (specifically the silicone sleeve or silicone layer) can be tailored to match skin colour so that this is not distracting. One issue with the hydrocarbon gel is that it is black if graphite is present or white is some other inorganic powders are used. The use of a silicone sleeve or silicone layer covering the hydrocarbon gel enables a more lifelike product to be developed through the use of pigments.

It is preferred if the sleeve contains less than 5 wt % water, especially less than 1.0 wt % water, especially is non-aqueous.

The thickness of the sleeve layer can be tailored to match requirements. Typically a sleeve may be 0.25 to 2.0 cm in thickness. Thus, in the embodiment below at all parts of the phantom, there is a sleeve thickness of 0.25 to 2.0 cm.

Ideally, the sleeve thickness is even all around the hydrocarbon gel layer.

In a second embodiment, the silicone layer can be tailored to match requirements. Typically a silicone layer may be 0.25 to 2.0 cm in thickness. Typically a housing or container may be 0.25 to 2.0 cm in thickness.

Overall, the phantom in these embodiments may have a thickness of may be 1.0 to 5.0 cm, such as 1.5 to 3.5 cm.

The phantom may have a length of may be 10 to 20 cm.

The phantom may have a width of may be 4.5 to 10 cm.

The surface area of the phantom can be varied as required. A suitable surface area is 30 to 100 cm².

The relative weights of the hydrocarbon gel layer and silicone sleeve or silicone layer can vary. Typically the weight ratio between the hydrocarbon gel layer and the sleeve layer or silicone layer is 1:3 to 3:1. The silicone layer is preferably in wt excess if this encases the hydrocarbon layer, such as 3:1 to 1:1 silicone to gel layer. In general, the silicone sleeve or silicone layer is used in as small amount as possible to serve its function of protecting the hydrocarbon gel layer.

The phantom is preferably provided with structures that mimic animal tissue. For example, tubes can be inserted through the phantom to mimic arteries and veins. Such tubes are conveniently made of silicone rubber. These tubes can be connected to external liquid sources so that fluid flows through the tubes. In this way fluid flow through the phantom can be monitored. These tubes may also be used with catheters or stents etc. to mimic real world situations. The tubes should be flexible in order to elicit the correct ultrasound response.

The phantom as a whole and/or tubes within the phantom may also be penetrated with needles to mimic a guided injection. A trainee ultrasonographer can therefore practice both an injection and then monitoring of that injection using ultrasound. In FIG. 6, we demonstrate that a needle can be detected in combination with the phantom of the invention. It will be appreciated therefore that the trainee ultrasonographer sees the silicone layer as a skin mimic. Needles do not pass through the housing.

The phantom can be provided with any structure that mimics a structure within the body which may need to be imaged. Other structures that might be present within the hydrocarbon gel layer include model cysts, or model tumour mass.

### Manufacture

The phantom may be manufactured in any convenient fashion, e.g. with two steps. A hydrocarbon gel block may be cast in the desired shape over any internal structures present (e.g. tubes for blood vessels, model cysts etc.). This block is then suspended in a two-part mould while the liquid components of the silicone are mixed. The silicone is degassed in a vacuum chamber to eliminate air content, then poured into the mould over the hydrocarbon gel block, fully encasing it. It can then be demoulded. If any tubes, tumours etc. are required in the phantom these are present at the start of the manufacturing process.

Alternatively, the hydrocarbon gel layer may be prepared within a suitable housing or container with any desired internal structures and a silicone layer applied thereover.

It may also be possible to cast a silicone sleeve, then to fill that sleeve with the molten gel after curing.

After demoulding, if tubes are present, connectors may be affixed to the tubes and a link made with a fluid source.

### Ultrasound

The ultrasound process can be carried out as normal. By emitting very high frequency sound waves into tissue and measuring the travel time of the returned echo, an ultrasound scanner can generate an image of underlying tissues. Ultrasound scanners used in medical ultrasonography are calibrated to work with human tissues. They thus assume certain standard values for their signal processing algorithms; for example, that the speed of sound will be approximately 1540 m/s. To work with existing, clinical ultrasound equipment, therefore, a phantom should be created from materials that emulate these properties to an adequate degree. It is preferred therefore if the phantom of the invention offers a speed of sound of 1400 to 1650 m/s.

Adjustment of the speed of sound within the phantom can be altered by changing the amount of inorganic powder present in the hydrocarbon gel layer and/or silicone sleeve. Control can also be affected though changing the nature of the gelling agent, the content of gelling agent within the hydrocarbon gel layer. In the sleeve, changing the glycerine content can affect the speed of sound.

The addition of inorganic powders results in an increase in echogenicity—the many, many transitions between the surrounding medium and the particulate medium each cause an echo to be reflected from the material boundary (this is a consequence of differences in acoustic impedance). Change in the speed of sound will affect the relative depth the ultrasound scanner will display, as well as potential deformations in the final image.

Adjustment of the speed of sound is primarily achieved via changing the density and stiffness of the materials. For the silicone, density may be affected by the addition of glycerine or other additives of different density to that of the silicone. In a preferred case, the silicone sleeve has a lower density than glycerine, so the addition of glycerine increases the density, increasing the speed of sound.

For the gel, density may be affected by density of the paraffin or mineral oil component used in manufacture (which are available in various densities). Additionally, the amount of gelling agent will affect the stiffness, also affecting speed of sound (in general higher density and higher stiffness both contribute to a higher speed of sound).

The invention will now be described with reference to the following non-limiting examples and figures.

### BRIEF DESCRIPTION OF THE FIGURES

FIG. 1 is a cross section of a phantom of the invention. The phantom comprises a silicone rubber sleeve (1) encasing a hydrocarbon gel layer (2). Passing through the phantom are silicone tubes (3) which mimic blood vessels. These tubes are connected to an external liquid source (not shown).

FIG. 2 is a photograph of a prepared phantom showing the exterior sleeve and tubes passing through the phantom.

In FIG. 3 the sleeve is cut open to reveal the hydrocarbon gel layer encased within the sleeve. The tubes pass through this hydrocarbon gel layer.

FIGS. 4, 5 and 6 are ultrasound images. FIG. 4 is an image of a real thigh showing the femoral artery and vein. FIG. 5 is an image using the phantom of the invention (FIG. 5). FIG. 6 is an in-line image (90 degrees out of cross section plane) of a guidewire being inserted into one of the vessels of the phantom.

FIG. 7 is a depiction of the moulding process. A mould body (1) encases a suspended hydrocarbon gel block (2) with a gap between mould body walls and the gel block to accept the silicone sleeve material. A tube (3) passes through the gel block (2) and mould body (1). The liquid silicone sleeve is poured from vessel (6) into the mould via sprue (4). A clamp (5) compresses the silicone sleeve to the hydrocarbon gel during the curing process.

FIG. 8 is a partial cross section of a phantom of the invention. The phantom comprises a silicone rubber sleeve layer (1) on top of a hydrocarbon gel layer (2). The hydrocarbon gel layer sits within a housing (7) (bottom not shown). The housing comprises a flange (8) to which the silicone layer adheres. As shown the silicone layer can wrap around the flanges to improve adhesion.

FIG. 9 is a photograph of a prepared phantom showing the housing and top silicone layer with tubes passing through the phantom.

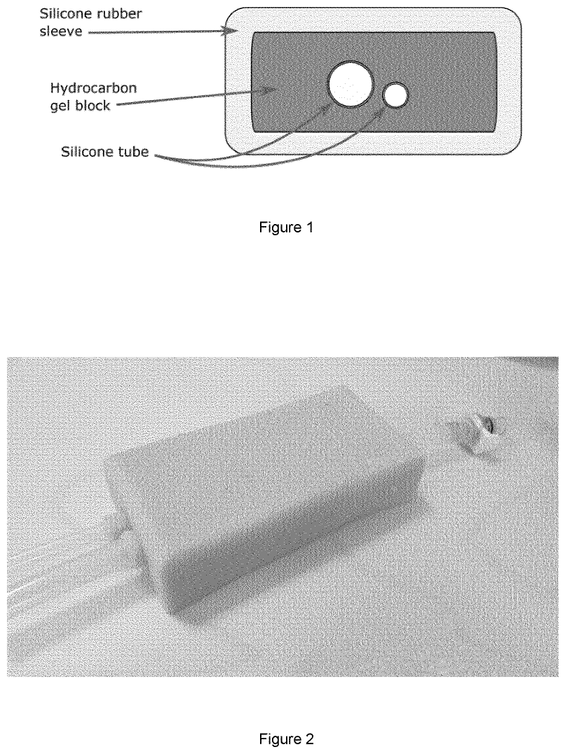

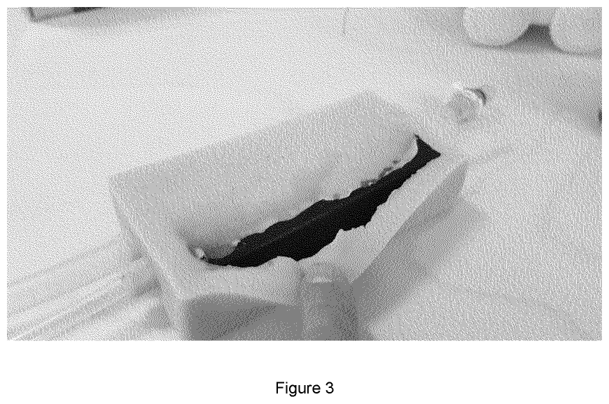

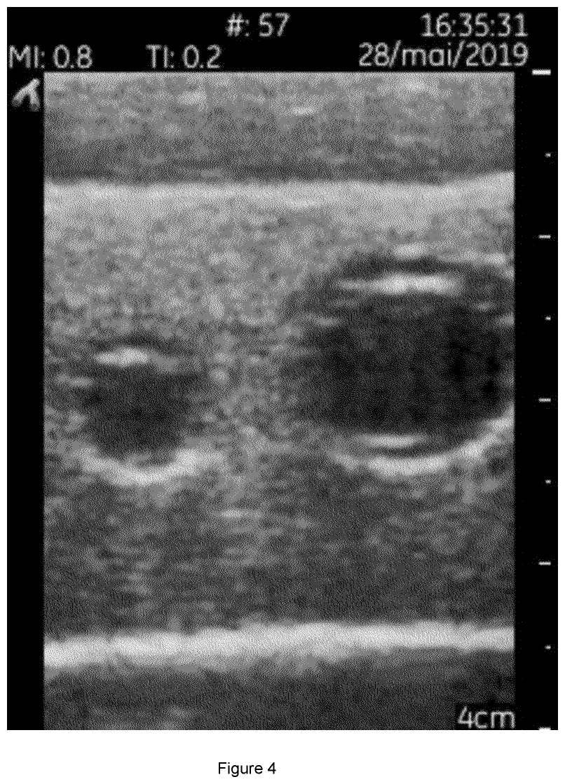

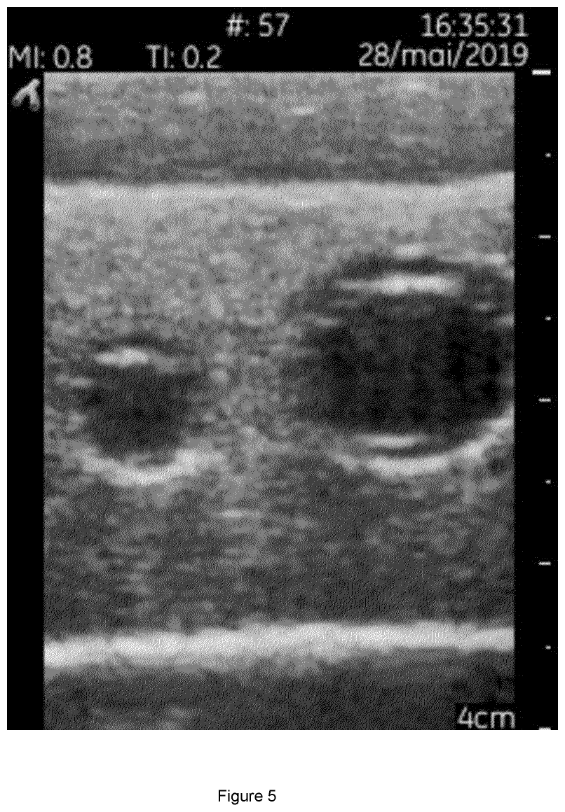

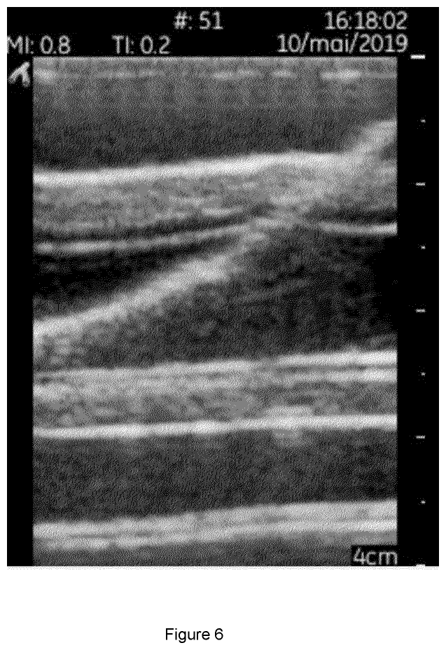

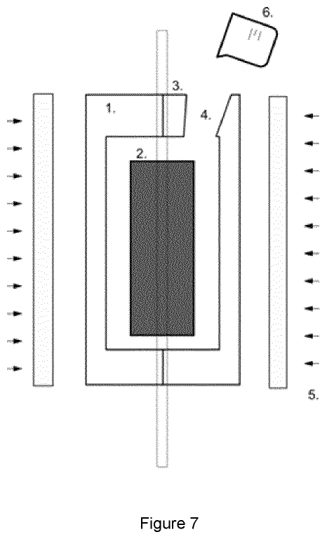

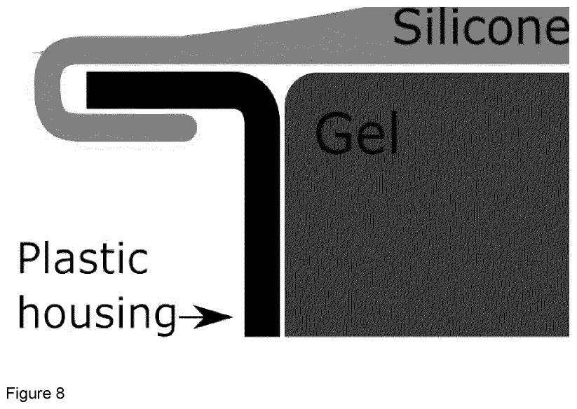

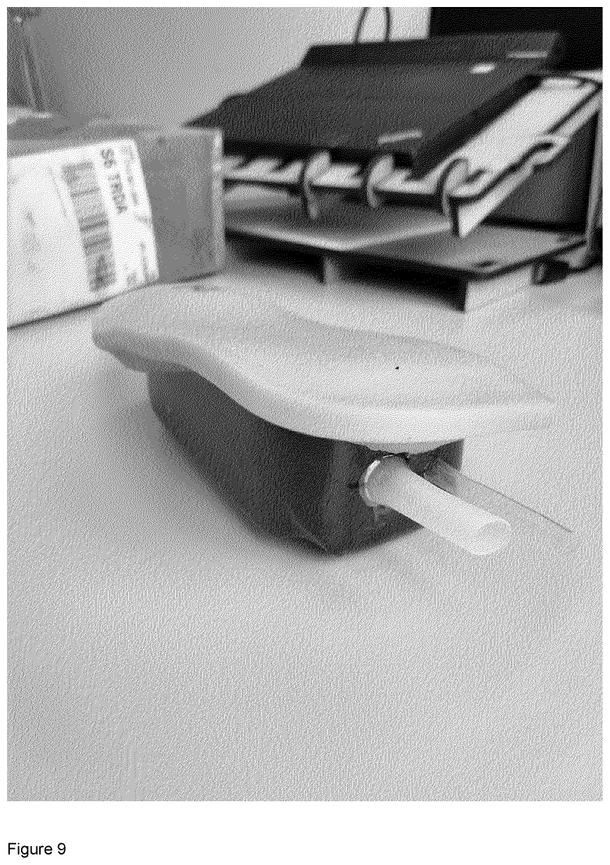

### EXAMPLE

Pre-mixed paraffin gel (consisting of approximately 95% wt paraffin and 5% wt gelling agent Penreco CP9000) was heated to melting. When molten, approx.1% wt graphite powder was added to the liquid. The liquid was then stirred thoroughly to disperse the powder.

The liquid paraffin gel mixture was poured into an open mould, encasing two silicone rubber tubes of exterior diameters 7 and 12 mm.

Once cooled and solidified, the resulting gel block with tubes was removed from the mould and placed in a two-part mould. The two-part mould has space between the gel lock and the insides of the mould, intended to be filled with silicone to form the sleeve.

The insides of this mould were prepared with a layer of petroleum jelly (Vaseline) to aid in de-moulding and a small amount of talc to give the cast surface a matte finish. This mould was then suspended vertically.

The two-component silicone (Eco-flex 00-10) was mixed, and a skin-tone pigment was added. This mixture was then degassed in a vacuum chamber (drawing approx. 30 in Hg for ˜5 minutes). The degassed silicone was then poured into the prepared mould and allowed to cure at room temperature for a period of four hours. The mould was opened, and connectors fixed to the tubes.

1. An ultrasound phantom comprising a hydrocarbon gel layer encased in a silicone sleeve, said hydrocarbon gel being doped with inorganic powder.

2. An ultrasound phantom comprising a hydrocarbon gel layer doped with inorganic powder disposed within an open container, such as a plastic container, said hydrocarbon gel layer having a lower surface in contact with the base of the container and an upper surface, wherein a protective silicone layer covers, such as entirely covers, the upper surface of said hydrocarbon gel layer.

3. An ultrasound phantom as claimed in any preceding claim wherein the hydrocarbon is paraffin or a mineral oil.

4. An ultrasound phantom as claimed in any preceding claim wherein the gelling agent in the hydrocarbon gel layer is a polystyrene/polyolefin copolymer.

5. An ultrasound phantom as claimed in any preceding claim wherein the hydrocarbon gel layer comprises 2 to 10 wt % gelling agent.

6. An ultrasound phantom as claimed in any preceding claim wherein the inorganic powder present in the hydrocarbon gel layer is alumina, titanium dioxide or graphite.

7. An ultrasound phantom as claimed in any preceding claim wherein there is 0.1 to 2.0 wt % inorganic powder present in the hydrocarbon gel layer.

8. An ultrasound phantom as claimed in any preceding claim wherein the silicone sleeve or silicone layer comprises glycerine or a glycol, e.g. 5 to 30 wt % glycerine or a glycol.

9. An ultrasound phantom as claimed in any preceding claim wherein the silicone sleeve or silicone layer comprises inorganic powder, e.g. 0.1 to 2.0 wt % inorganic powder.

10. An ultrasound phantom as claimed in any preceding claim wherein the silicone sleeve or silicone layer comprises pigment to give it the colour of skin.

11. An ultrasound phantom as claimed in any preceding claim wherein one or more tubes pass through the phantom.

12. An ultrasound phantom as claimed in any preceding claim which is cuboidal or shaped like a body part.

13. An ultrasound phantom as claimed in any preceding claim having a thickness of 1.0 to 5.0 cm.

14. An ultrasound phantom as claimed in any preceding claim which is non-aqueous.

15. An ultrasound phantom comprising a hydrocarbon gel layer comprising paraffin or a mineral oil and a polystyrene/polyolefin block copolymer completely encased in a silicone sleeve, said hydrocarbon gel comprising alumina, titanium dioxide or graphite powder and said silicone sleeve comprising an inorganic powder and glycerine.

16. Use of a phantom as claimed in claims 1 to 15 in ultrasonography and/or as a mimic for needle insertion.

17. A process for the preparation of an ultrasound phantom as claimed in claims 1 to 15 comprising preparing a hydrocarbon gel doped with inorganic powder and encasing the same in a silicone sleeve.
